(19) United States
(12) Patent Application Publication
(10) Pub. No.: US 2014/0100603 A1
(43) Pub. Date: Apr. 10, 2014

(54) DEVICE FOR SEALING PERFORATIONS AND SUSTAINING FLOW

(71) Applicants: Dan Aravot, Kazerin (IL); Ehud Almon, Kiryat Tivon (IL); Eran Hirszowicz, Ramat Gan (IL); Hila Yaron-Brauman, Tel-Aviv (IL)

(72) Inventors: Dan Aravot, Kazerin (IL); Ehud Almon, Kiryat Tivon (IL); Eran Hirszowicz, Ramat Gan (IL); Hila Yaron-Brauman, Tel-Aviv (IL)

(21) Appl. No.: 13/668,736

(22) Filed: Nov. 5, 2012

Related U.S. Application Data
(63) Continuation of application No. PCT/IB2010/051984, filed on May 5, 2010.

(51) Int. Cl. A61B 17/00

(52) U.S. Cl. CPC A61B 17/0057
USPC 606/213

(57) ABSTRACT

A device for sealing perforations and sustaining a flow of liquids as well as a method of using the same are disclosed. The device can be beneficially employed in emergency trauma care or for performing a rapid aortic cannulation procedure or other catheterizations. The device includes a catheter member, a mandrel member, a cannula member and a sealing element.

### CROSS REFERENCE TO RELATED APPLICATIONS

The present application claims the benefit of priority to U.S. Provisional Patent Application Ser. No. 61/175,470 filed May 5, 2009, entitled “CARDIOVASCULAR SEALER AND FILLER” and U.S. Provisional Patent Application Ser. No. 61/296,058 filed Jan. 19, 2010, entitled “DEVICE FOR SEALING PERFORATIONS AND SUSTAINING FLOW”; the aforementioned applications are incorporated herein by reference in their entirety.

### FIELD OF THE INVENTION

The present invention relates to medical devices in general. More particularly, the present invention relates to devices capable of sealing perforations, inter alia within the cardiovascular system, and sustaining a flow thereto/therefrom.

### BACKGROUND ART

One of the main causes of death in trauma injuries is haemorrhage from the major blood vessels of the torso or the heart. If not efficiently treated without delay, the haemorrhage causes exsanguination and may lead to death within minutes. Exsanguination is the estimated main cause in 5-10% of the death toll of urban trauma injuries and 10% or more in military trauma injuries.

Contradistinctively to the limbs, whereon a tourniquet or direct pressure can be employed in order to efficiently halt a haemorrhage, perforations or ruptures in the major blood vessels of the torso or the heart itself cannot be treated in either of these manners. Furthermore, if the rupture occurs near the bifurcation of a limb from the corpus, such as for instance rupture in the femoral artery at the groin region, a tourniquet cannot be used and a direct pressure may fail to achieve the desired result. Moreover, whenever a tourniquet is applicable, if employed for a prolonged time period, the accompanying interruption of blood circulation in the limb may occasionally lead to necrosis of the limb tissues and necessitate amputation.

A device for sealing perforations by discretely blocking the perforation itself, from inside and outside of the vessel's wall around the perforation, rather than by means of a direct or radial pressure and complete cave-in of the blood vessel would be applicable in treating perforations or ruptures of the major blood vessels of the corpus and of the heart. Such a device would be specifically beneficial in treating haemorrhages associated with the blood vessels of the thoracic, abdominal and pelvic sections of the corpus such as the aorta, abdominal aorta, vena cava, pulmonary vein, thoracic aorta, etc. Moreover, such a device would also constitute a preferred alternative for treating haemorrhages of blood vessels of the limbs, allowing halting a haemorrhage and maintaining blood circulation in the limb.

A specific class of trauma is the substantial haemorrhage from injured major blood vessels of the corpus or the heart. This is typically accompanied by hypovolemia which requires a rapid restitution of the volume of circulating blood in the body. Pressure infusion devices (PIDs) such as various sleeves, jackets, pumps and the device commercialized under the tradename of Level 1 (Level 1 Technologies, Inc., Rockland, Mass.) are widely used to maintain normovolemia in patients experiencing large-volume blood loss. Whereas the prevailing, routinely used PIDs can infuse liquids with flow rates of up-to mL/min, the Level 1 devices can uphold flow rates of up to 500 mL/min. Such flow rates may nevertheless be insufficient for effective treatment of severe trauma injuries. High-Flow systems, employing cannulas with diameter of up to 10 mm, such as the High-Flow Blood Filter (Saftifilter Blood Administration Sets; Cutter Biological, Berkeley, Calif.) can sustain flow rates of up to ˜600-1500 mL/min; however, these systems are not portable and thus typically cannot be used for treatment of trauma conditions. A portable, handy device applicable in emergency field-care of injury incidents and capable of infusing liquids with flow rates of more than 500 mL/min, would thus have a specific benefit in emergency treatment of trauma conditions.

U.S. Pat. Nos. 7,169,176, 7,011,679, 6,976,952, 6,740,111, 6,635,080, 4,747,848, 4,545,082, 6,368,347, 6,352,554 and 6,264,662 are believed to represent the current state of the art.

### SUMMARY OF THE INVENTION

In accordance with some embodiments of the present invention a device capable of sealing perforation/s within and sustaining a flow into or out of an organ and a method of using the same are provided. The device is specifically beneficial in emergency trauma care of perforations or raptures in the cardiovascular system. The device can be beneficially employed for performing a quick aortic cannulation procedure or other catheterizations.

The device of the present invention is a dual-purpose tool. Each one of its functions is associated with specific structural features, as will be described below in some detail. The object of the first function is to seal perforations, e.g. in the cardiovascular system caused by trauma injuries and characterized by a substantial haemorrhage. The object of the second function is to sustain effective amounts of liquid flow into the sealed organ or the drainage of fluids therefrom; in the case of cardiovascular system, in order to avoid possible hypovolemic shock caused by the haemorrhage, thereby stabilizing the condition of the patient.

### BRIEF DESCRIPTION OF THE DRAWINGS

The present invention will be understood and appreciated more fully from the following detailed description taken in conjunction with the appended drawings in which:

FIG. 1A is an isometric view of an embodiment of the device for sealing perforations and sustaining a flow of liquids;

FIG. 1B is an exploded isometric view of a device for sealing perforations and sustaining a flow of liquids;

FIG. 2A is an enlarged isometric view of the anterior portion the catheter member and the expandable flange, in sprawled or unfolded conformation, of the device shown in FIGS. 1A and 1B;

FIG. 2B is an enlarged cross-sectional view of the anterior portion the catheter member and the expandable flange, in sprawled or unfolded conformation, of the device shown in FIGS. 1A and 1B;

FIG. 2C is an enlarged cross-sectional view of the expandable flange, in contacted or folded conformation, disposed within the confining sleeve;

FIG. 3A is an isometric view of the cannula member, of the device shown in FIGS. 1A and 1B;

FIG. 3B is an isometric view of the pulling tab of the cannula member shown in FIG. 3A;

FIG. 3C is an isometric view of the confining sleeve of the cannula member shown in FIG. 3A;

FIG. 3D is a side view of the confining sleeve of the cannula member shown in FIG. 3A;

FIG. 4A is an isometric view of the sealing element, of the device shown in FIGS. 1A and 1B;

FIG. 4B is an exploded isometric view of the sealing element, of the device shown in FIGS. 1A and 1B;

FIG. 4C is a cross-sectional view of the toroidal cushion of the sealing element shown in FIGS. 4A and 4B;

FIG. 5 is an isometric view of a preferred embodiment device for sealing perforations and sustaining a flow of liquids;

FIG. 6A is a cross-sectional view of the posterior portion of the device for sealing perforations and sustaining a flow of liquids shown in FIG. 5;

FIG. 6B is an enlarged cross-sectional view of the locking mechanism of the device shown in FIG. 6A, in inactive configuration;

FIG. 6C is a cross-sectional view of the posterior portion of the device for sealing perforations and sustaining a flow of liquids shown in FIG. 5;

FIG. 6D is an enlarged cross-sectional view of the locking mechanism of the device shown in FIG. 6C, in active configuration;

FIG. 7A is an isometric view of configuration 300A of an embodiment of the device of the present invention adapted for sealing perforations in globular or apical organs;

FIG. 7B is an isometric view of configuration 300B of an embodiment of the device of the present invention adapted for sealing perforations in globular or apical organs;

FIG. 7C is an isometric view of configuration 300C of an embodiment of the device of the present invention adapted for sealing perforation in globular or apical organs;

FIG. 8 is an isometric view of yet another embodiment 400 of the device of the present invention adapted for sealing perforation in globular or apical organs.

### DISCLOSURE OF THE INVENTION

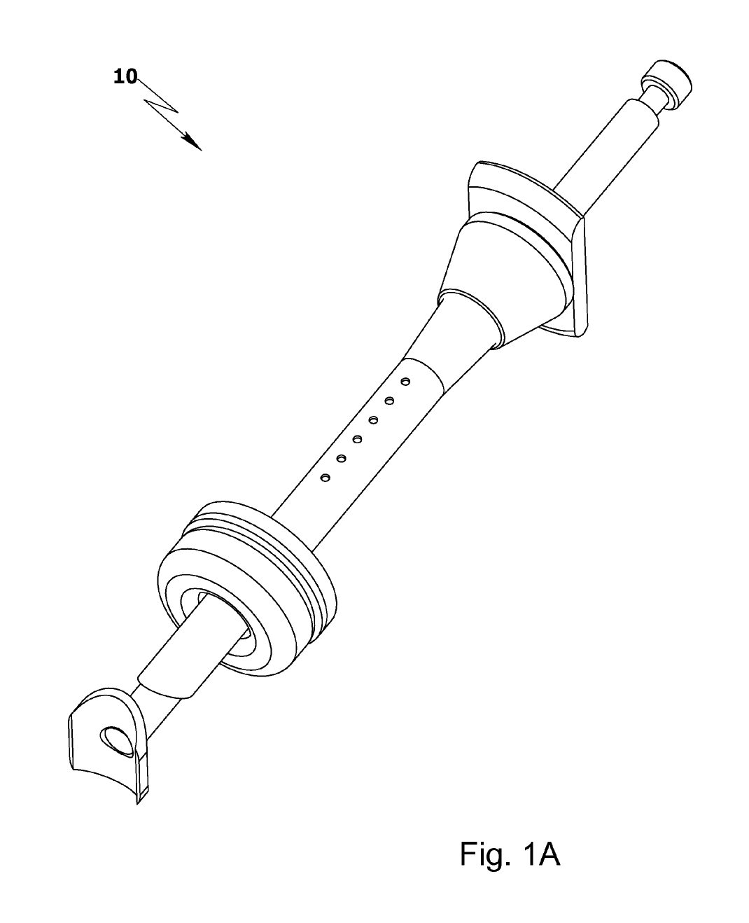

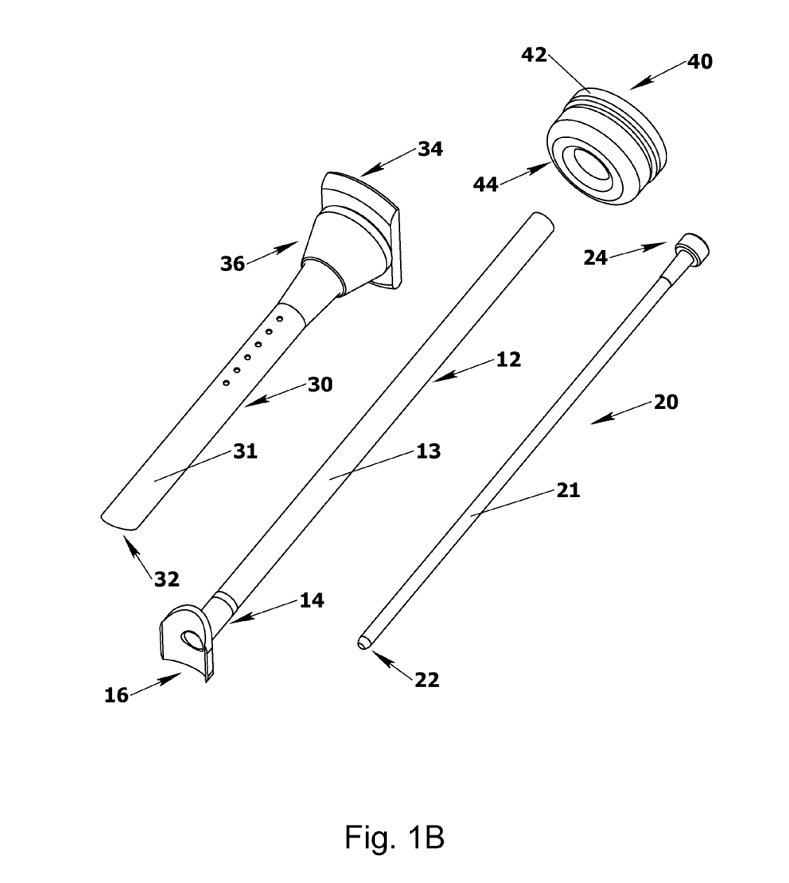

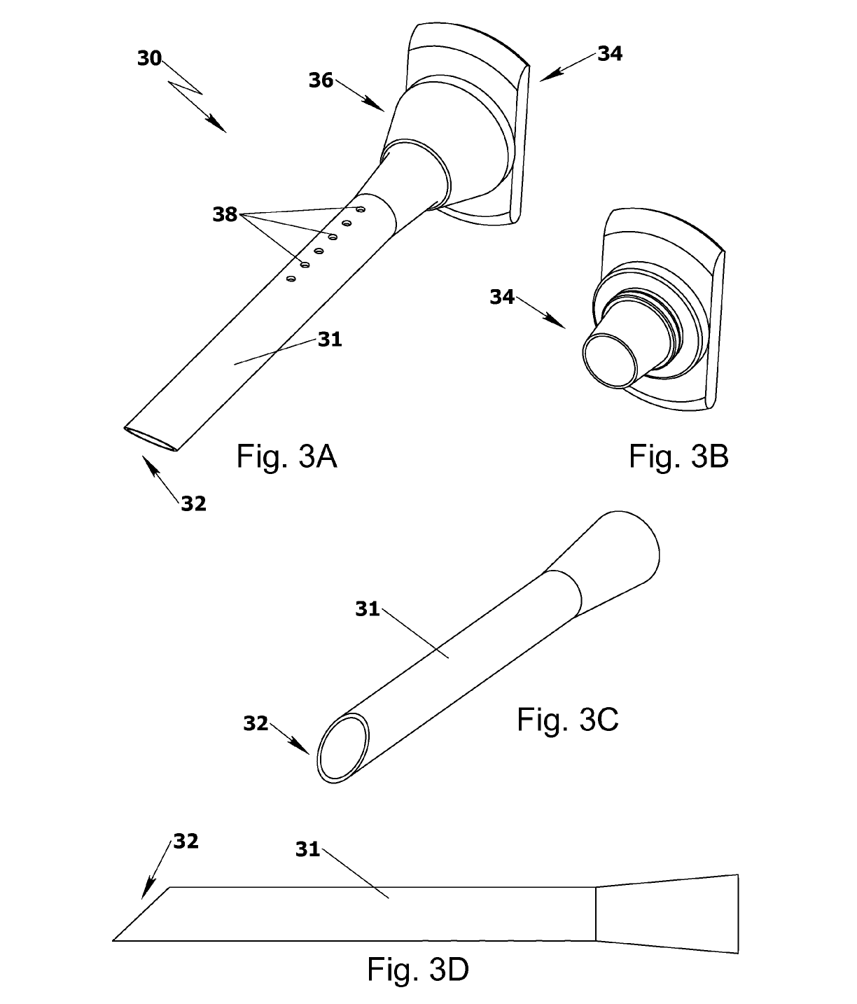

Reference is now made to FIGS. 1 to 4, showing device 10 for sealing perforations and sustaining a flow of liquids (hereinafter DSPSFL). DSPSFL 10 is adapted for sealing perforations in essentially tubular organs or tracts, e.g. various organs of the cardiovascular system and particularly blood vessels. DSPSFL 10 comprises catheter member 12, mandrel member 20, cannula member 30 and sealing element 40.

Catheter member 12 comprises sheath element 13, furnished with thin-walled neck portion 14 towards distal end, and terminates with expandable flange 16 at the distal or otherwise anterior end; the end of member 12 opposite flange 16 is referred to hereinafter as proximal or posterior. Mandrel member 20 comprises core shaft 21, distal tip 22 and proximal cap 24. Cannula member 30 comprises confining sleeve 31, terminated by tip 32, pulling tab 34 and interconnecting element 36. Sealing element 40 comprises bushing 42 and toroidal cushion 44.

Sheath element 13 comprises interior lumen 13L extending therethrough. The outer diameter of sheath element 13 typically ranges from about 3 mm to about 8 mm but the range of about 6 to 7 mm is preferable. The inner diameter of sheath element 13 typically ranges from about 1.5 mm to about 7 mm but the preferable range is about 4.5 to 6 mm. Sheath element 13 is preferably made of biocompatible elastomeric material, such as silicone rubber or soft PeBax®. The material that sheath element 13 made of is preferably characterized by Shore hardness of about 60 units, on the A-type D2240 ASTM durometric scale, and elongation ability of about 350%, before reaching the yield point.

The outer diameter of neck portion 14 typically equals the outer diameter of sheath element 13. The thickness of the wall of neck portion 14 is preferably less than 1 mm. Neck portion 14 is preferably characterized by Shore hardness of about 40 units, on the A-type D2240 ASTM scale, and elongation ability of about 750%, before reaching the yield point. Provided that sheath element 13 and neck portion 14 thereof are made of different materials various methods known in the art may be employed to form the integral structure of catheter-like member 12, inter alia including co-molding and conjugation. The distal portion of sheath element 13 or neck portion 14 of member 12 may be furnished with notches, recesses or grooves to facilitate affixing of sealing element 40 at the anterior of sealing element 40, as will be elaborated infra.

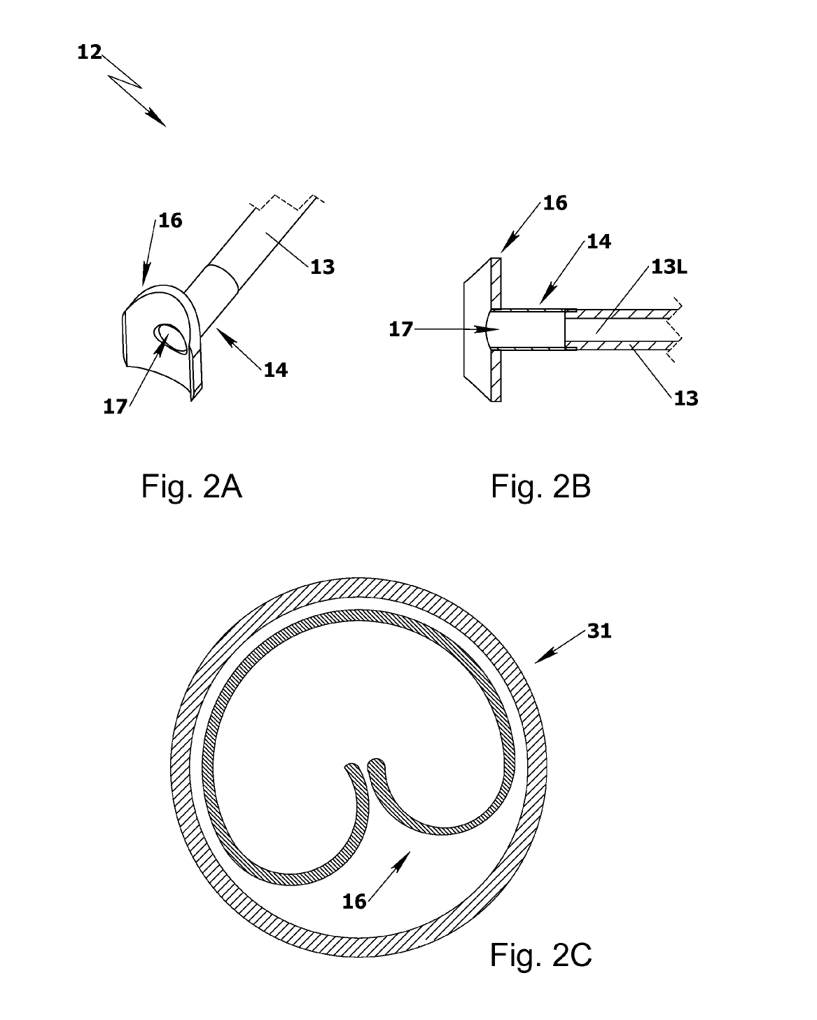

In some embodiments expandable flange 16 is characterized by a trough-shaped configuration, with chamfered or rounded edges and/or corners. The convex surface of expandable flange 16 is adapted to adjoin the interior surface of a blood vessel having an essentially tubular shape. The width of expandable flange 16 typically ranges from 10 mm to 15 mm and lengths the form 15 mm to 30 mm. Referring now to FIG. 2A, expandable flange 16 comprises aperture 17, at the centre thereof, forming a continuum with interior lumen 13L of sheath element 13. Aperture 17 is also referred to hereinafter as the outlet of DSPSFL 10; whereas the aperture at the proximal end of sheath element 13 is referred to as the inlet thereof.

It should be acknowledged that expandable flange 16 of a trough-shaped configuration and the sizes set forth supra is merely an exemplary element of an embodiment of the invention; whereas the expandable elements to be used with catheter-like member of the present invention may assume different shapes and a variety of sizes, mainly dependent upon the medical application in which the DSPSFL is employed and the organ on which the DSPSFL is implemented, as will be described below in some detail.

Expandable flange 16 is capable of assuming, inter alia, a contracted (folded) or an expanded (sprawled or unfolded) conformations. In some examples, expandable flange 16 is made entirely of a resilient material that tends to inherently expand when contracted, due to the intrinsic bias thereof. In other examples, expandable flange 16 is made of a less biasing material typically furnished with structural framework or scaffolding (not shown), for instance embedded at the rims thereof, which confer the desired intrinsic bias and the expandability to the expandable flange.

The conformation of expandable flange 16 shown in FIGS. 2A and 2B is the expanded or sprawled one; whereas in the contracted or folded conformation flange 16 is accommodated within confining sleeve 31. Expandable flange 16 may be folded up, for instance so that the flanking portions thereof are folded vis-à-vis each other, as schematically shown in FIG. 2C. Any other contracted or folded conformation of expandable flange 16 may be employed to facilitate the accommodation thereof within confining sleeve 31. In contracted conformation flange 16 confined by sleeve 31; thereby the expansion of the former is precluded by the latter. In contracted conformation the effective exterior diameter of DSPSFL 10 is the outer diameter of sleeve 31.

Expandable flange 16 may further include at least one structural element (not shown), in a non-limiting manner including slot, aperture, tab or pocket, dedicated for engagement with tip 22 of mandrel 20; whereby upon manipulating/advancing mandrel 20, expandable flange 16 is respectively manipulated/advanced. The aforementioned structural element is typically disposed on the concave surface of expandable flange 16.

Mandrel member 20 comprises core shaft 21. Core shaft 21 is an essentially elongated cylindrical body, made of a rigid material, e.g. stainless steel and/or polymeric material. Core shaft 21 is insertable into lumen 13L and used to confer structural firmness to sheath element 13; thereby allowing manipulating/advancing of DSPSFL 10.

Mandrel member 20 comprises tip 22 at the distal end thereof. Tip 22 may include at least one structural element (not shown), in a non-limiting manner including slot, aperture, tab or pocket, dedicated for engagement with a respective structural element (not shown) of expandable flange 16.

Mandrel member 20 further comprises cap 24 at the proximal end thereof. Cap 24 can be shaped like a knob and/or furnished with texture to facilitate a more ergonomic and convenient grip thereof; thereby facilitating manipulation/advancement of DSPSFL 10 by the operator.

In some embodiments shaft 21 comprises a lumen (not shown) extending therethrough and tip 22 includes an aperture at the anterior end allowing the access of blood thereto. Cap 24 is transparent or at least translucent and comprises an interior lumen (not shown) forming a continuum with the lumen of shaft 21 (not shown). By this means, upon placement of DSPSFL 10 inside a blood vessel, tip 22 is exposed to the arterial blood pressure and the blood fills the lumen of shaft 21 (not shown) eventually partially occupying the interior of cap 24; whereby the presence of the tip of DSPSFL 10 in a blood vessel can be visually detected (serving as a blood indicator), much in the same way as caps of the needles of venous cannulae commercialized under the tradename Venflon®.

Cannula member 30 comprises confining sleeve 31. Sleeve 31 terminates with slanted distal tip 32. The outer diameter of sleeve 31 typically ranges from about 4 mm to about 10 mm; preferably from about 7 mm to 8 mm. The inner diameter of sleeve 31 typically equals the outer diameter of sheath element 13 or slightly larger so as to establish a contiguous sliding of the former along the latter. Sleeve 31 is preferably made of a stiff biocompatible polymeric material, such as PTFE (Teflon®), PeBax®, Nylon or a combination of several materials. Distal tip 32 is preferably shaped and sized as the tips of aortic cannulae known in the art. Distal tip 32 may be made of an elastomeric material softer than sleeve 31. Pulling tab 34 is secured by interconnecting element 36 to sleeve 31; thereby upon pulling tab 34, sleeve 31 is retracted therewith.

Sleeve 31 of cannula member 30 confines expandable flange 16, thereby precluding the unfolding, sprawling or expansion of the latter, thus retaining it in the contracted or folded conformation. Cannula member 30 is preferably designed essentially as aortic cannulae and adapted to facilitate the ease of manipulation thereof by the operator, so as to be conveniently placed within an organ, such as blood vessel or the heart, through a perforation or incision therein. Cannula member 30 may further include graduations or indicia 38, to indicate the depth of the penetration into a perforation, incision or wound and the orientation of inclination of slanted tip 32 at the distal end of confining sleeve 31 while therein.

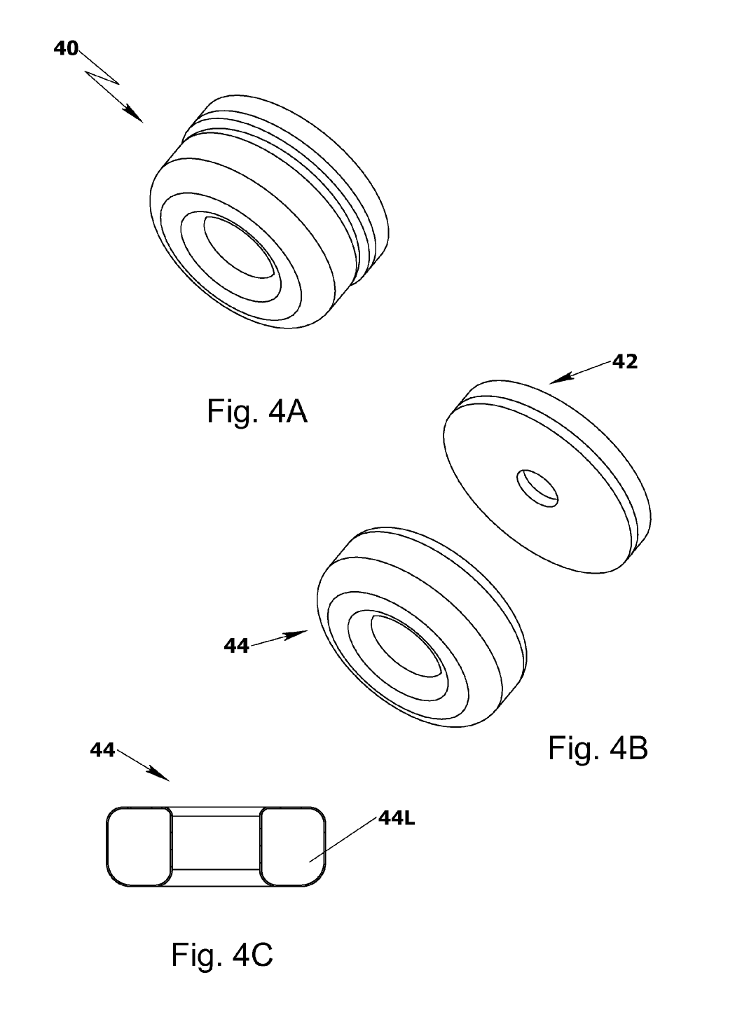

Referring to FIGS. 4A-C, sealing element 40 comprises bushing 42 and anteriorly disposed toroidal cushion 44. Bushing 42 is contiguously slidable along catheter member 12. The circumference of bushing 42 can be ergonomically shaped and/or furnished with texture to facilitate en enhanced grip thereof. Bushing 42 may further include a catches or detents (not shown), to be affixed thereby to the proximal end of catheter member 12, upon interaction with the respective notches, recesses or grooves at the distal portion of sheath element 13 or neck portion 14 of member 12, as were mentioned supra. The outer diameter of toroidal cushion 44 typically ranges from about 15 mm to about 35 mm and the thickness ranges from about 10 mm to about 20 mm. Toroidal cushion 44 comprises balloon-like lumen 44L. Toroidal cushion 44 is preferably made of an elastomeric biocompatible material, such as silicone rubber. Lumen 44L is filled with biocompatible gel of about 3 to 10 Shore hardness units, on the A-type D2240 ASTM durometric scale, such as silicone gel MED2-6300 available from NuSil, at 1105 Arlington Drive Toms River, N.J. 08755 USA. In some examples lumen 44L is filled with gaseous substance, e.g. air.

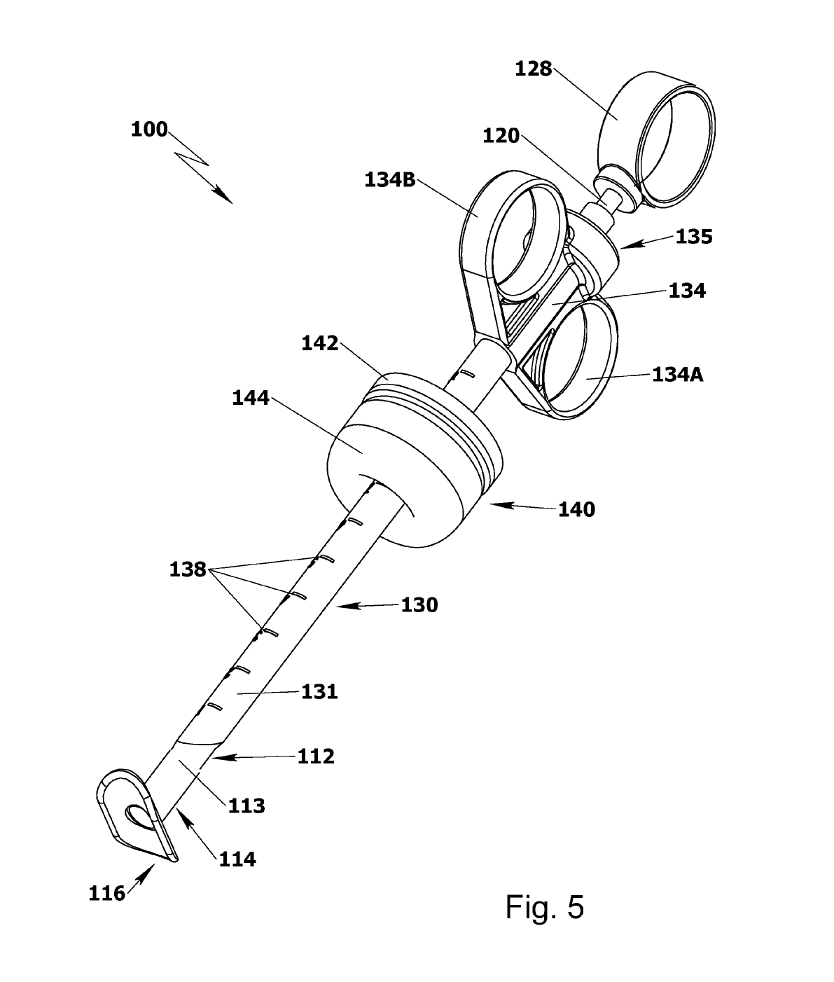

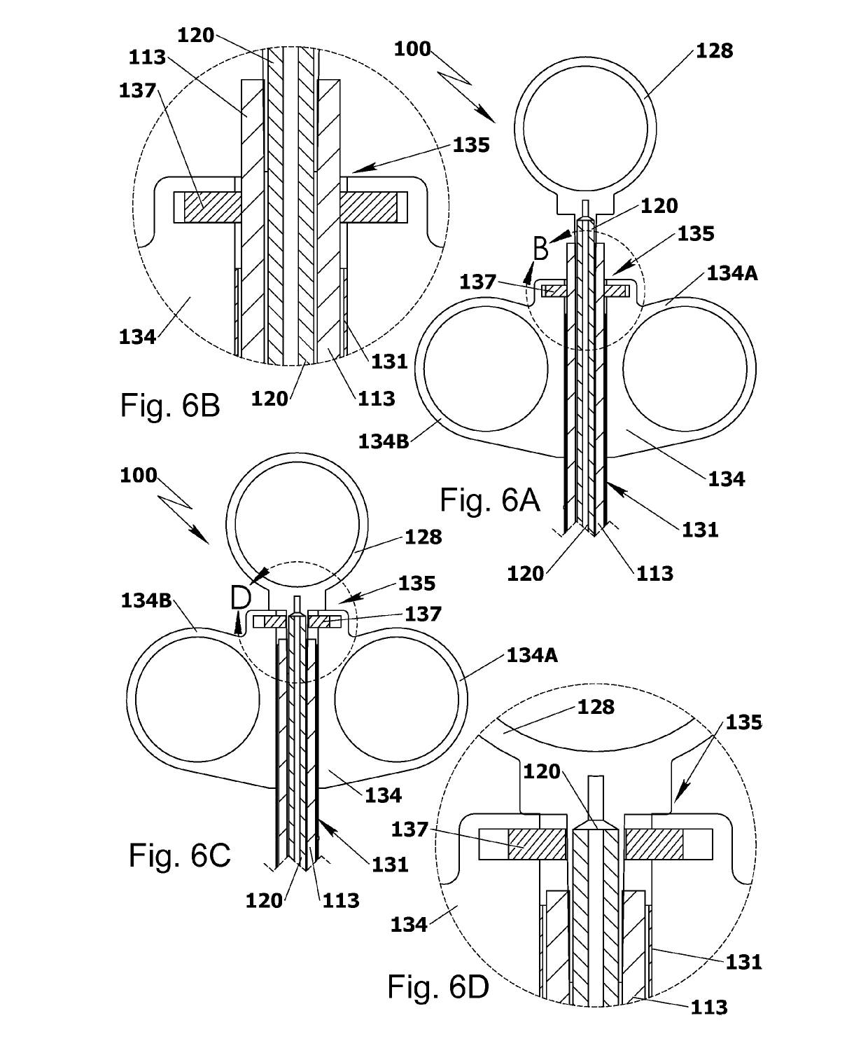

Reference is now made to FIGS. 5 to 6, showing DSPSFL 100. DSPSFL 100 is adapted for sealing perforations in essentially tubular organs, e.g. blood vessels. DSPSFL 10 comprises catheter member 12, mandrel member 20, cannula member 30 and sealing element 40.

Catheter member 112 comprises sheath element 113, furnished with thin-walled neck portion 114 towards distal end, and terminates with expandable flange 116. Mandrel member 120 comprises proximal thumb-hold tab 128. Cannula member 130 comprises confining sleeve 131 including indicia or graduations 138 and terminating with a slanted tip, pulling element 134 that includes finger-hold tabs 134A and 1348 and mandrel interlocking mechanism 135. Sealing element 140 comprises bushing 142 and toroidal cushion 144. The aforementioned parts of DSPSFL 100 are characterized by reference to their respective equivalents and counterparts of DSPSFL 10, unless otherwise indicated.

The proximal thumb-hold tab 128 of mandrel member 120 and finger-hold tabs 134A and 1348 of pulling element 134 facilitate an enhanced ease of operation of DSPSFL 100. Finger-hold tabs 134A and 1348 particularly provide for posteriorly retracting pulling element 134 and confining sleeve 131 of the cannula member 130 over catheter member 112, while concomitantly stabilizing mandrel 120 by grasping thumb-hold tab 128, which is convenient for operating DSPSFL 100, as elaborated below.

Mandrel interlocking mechanism 135 of cannula member 130 comprises annular locking element 137. Locking element 137 is a split-ring or -washer or spring-washer or any other element capable of being biased towards small internal diameter. Interlocking mechanism 135 is employed to facilitate the removal of mandrel 120 from the interior lumen of sheath element 113 while concomitantly stabilizing catheter member 112 thus preventing any forceful pulling of sheath element 113 and particularly of expandable flange 116. In inactive configuration annular locking element 137 is disposed around sheath element 113 contiguously slidable along the exterior surface thereof, as shown in FIG. 6B. Upon translation of pulling element 134 in the posterior direction relatively to sheath element 113, annular locking element 137 is slid over the exterior surface of the latter, until reaching the proximal end thereof. Thereupon annular locking element 137 spontaneously contracts to assume a diminished internal diameter, i.e. active configuration, shown in FIG. 6D. In active the configuration, annular locking element 137 is disposed around mandrel 120 thereby effectively preventing posterior translation of sheath element 113 beyond annular locking element 137 and away from pulling element 134. Accordingly, the operator can pull mandrel 120 from the interior lumen of sheath element 113, for instance by applying a manual force to thumb-hold tab 128 in the posterior direction, while preventing translation of sheath element 113 in the posterior direction, by stabilizing pulling element 134, for instance by grasping finger-hold tabs 134A and 134B.

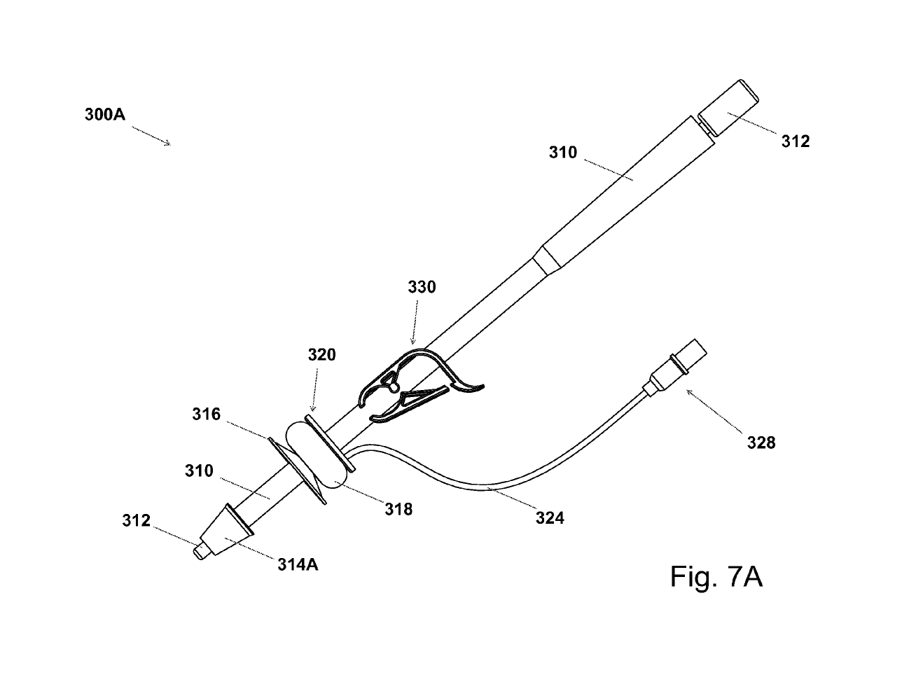

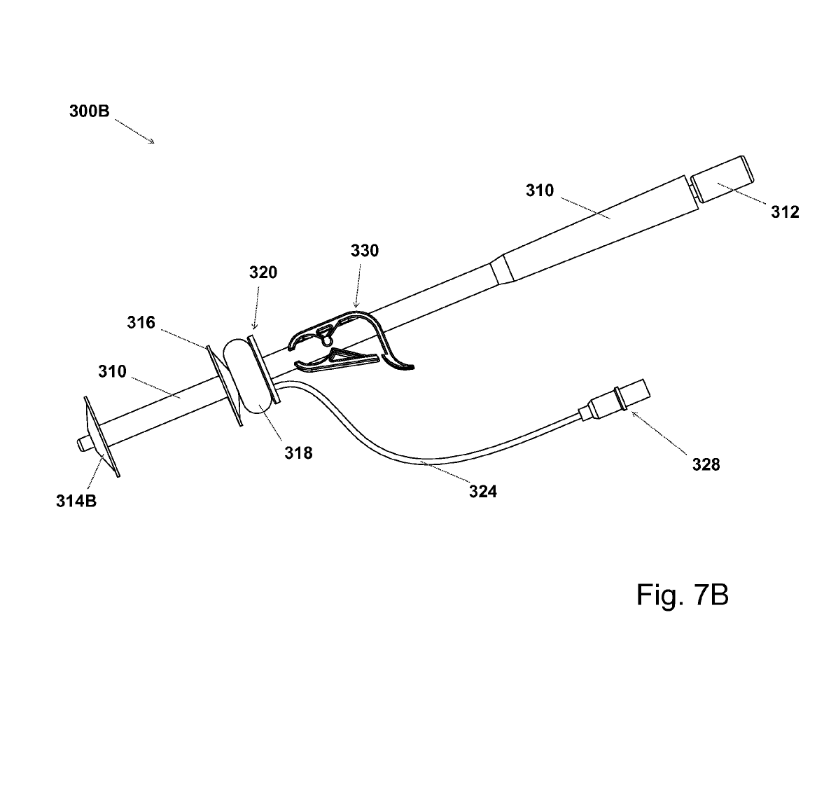

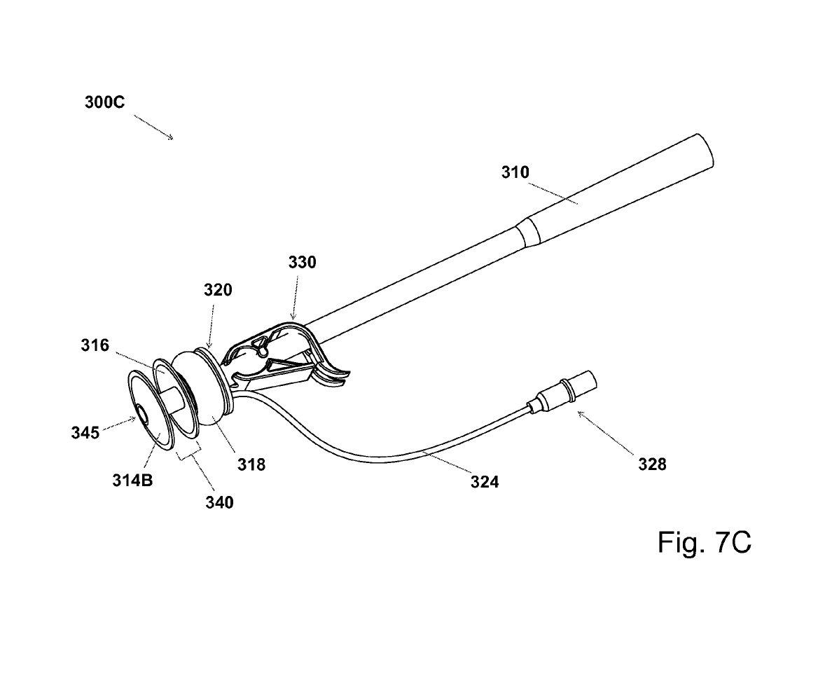

The embodiments of the DSPSFL shown throughout FIGS. 1 to 6 are particularly adapted for sealing perforations in an essentially tubular organ, such as blood vessel. The method of operating the DSPSFL will be explained in more details below, by explaining in same detail the operation of DSPSFL implemented on globular or apical organs. To elaborate structural and functional feature of DSPSFL embodiments adapted for sealing perforations in an essentially globular or apical organ, e.g. the heart, reference is now made to FIGS. 7A to 7D showing DSPSFL embodiments 300A-300C. Within the interior lumen of catheter member 310, mandrel 312 is disposed, its ends shown extending throughout the entire length of catheter member 310, in order to confer a required degree of firmness to DSPSFL 300A; thereby providing for conveniently manipulating expandable flange 314A at the distal end of catheter member 310 through a perforation in the cardiovascular system and in the heart. The interior lumen of catheter portion 310 is designed to allow sufficient amounts of liquids to pass through in a relatively short period of time and characterised by flow rates of at least 0.25-0.5 L/min. Mandrel 312 may be furnished with a blood indicator, as denoted above. Expandable flange 314A retained in the contracted conformation by means of a cannula member (not shown) comprising a confining sleeve (not shown) threaded thereon and preventing expandable flange 314A from unfolding into the expanded or sprawled conformation 314B. The cannula member (not shown) and the confining sleeve (not shown) are characterized as cannula member 30 and sleeve 31 shown in FIGS. 1 to 4. Expandable flange 314A may be folded in the confining sleeve (not shown) in an umbrella-like manner. It should be acknowledged that the orientation of expandable flange 314A folded in the proximal direction is merely exemplary; whereas a folding of expandable flange 314A in the distal direction is equally applicable and may be preferred.

In some embodiments expandable flange 314A is furnished with a scaffolding, such as a biasing string (not shown) threaded into a circumferential channel (not shown) embedded in the expandable flange 314A; thus conferring the desired intrinsic bias and expandability to flange 314A. Expandable flange 314A may further include at least one structural element (not shown), in a non-limiting manner including slot, aperture, tab or pocket, dedicated for engagement with the tip of mandrel 312; whereby upon of mandrel 312 expandable flange 314A is respectively manipulated/advanced, similarly to what has been described hereinabove.

Proximally to expandable flange 314A, on catheter member 310, mitral washer 316, inflatable toroidal cushion 318 and bushing 320 are disposed, being contiguously slidable therealong, collectively forming the sealing element of the DSPSFL. The inflatable lumen of toroidal member 318 forms a continuum with conduit 324 which is connected to inflator unit 328. Examples of inflator unit 328 include: check valves, baffles, Luer tip actuated valves, Luer tip connectors, manually powered pumps, non-manually powered pumps, a pressurized gas tank and various combinations thereof. Above mitral washer 316, inflatable toroidal cushion 318 and bushing 320 on catheter portion 310 clamp 330 is disposed, providing for contiguously driving the aforementioned elements along catheter portion 310 towards the expandable flange 314A and securing them at the distal end thereof. Clamp 330 also provides for controlling the flow maintained through the interior lumen of catheter portion 310.

Inflatable toroidal cushion 318 comprises a balloon-like lumen facing the biological tissue surrounding the perforation. This balloon-like lumen can be inflated, once the sealing element has been put in place, in order to create increased and more uniform pressure on the biological tissue surrounding the perforation thus facilitating more an efficient anastomosis.

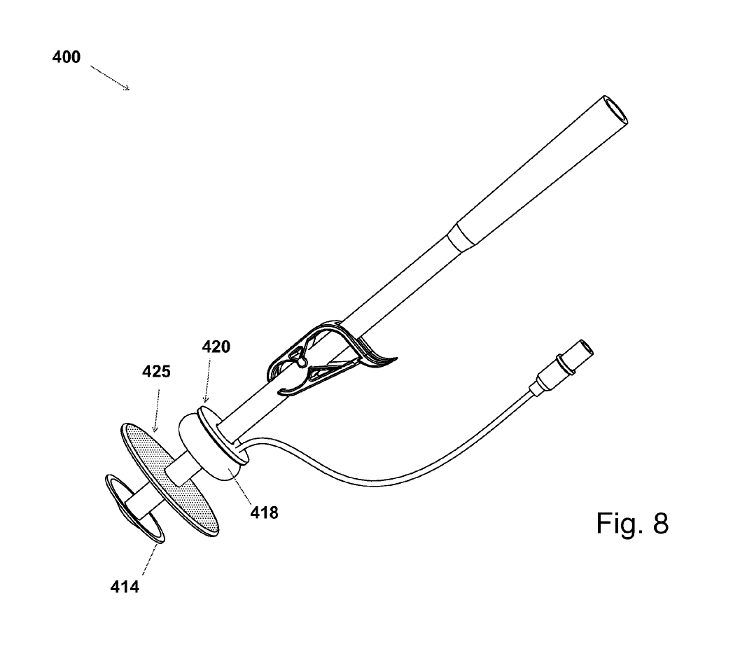

Reference is now made to FIG. 8 wherein another referred embodiment 400 of the DSPSFL of the present invention is shown. Embodiment 400 is in essence similar to the embodiment shown throughout FIGS. 7A-7C, having similar expandable flange 414 and sealing element formed by inflatable toroidal cushion 418 and bushing 420. An absorbent patch 425 is disposed in-between expandable flange 414 and expandable flange. Absorbent patch 425 can be covered, impregnated, embedded with or somehow otherwise contain either of the following agents: a super absorbent polymer (SAP); a coagulating and/or vasoconstrictive agent/s; an antiseptic, antibacterial or antifungal agent/s; anti-adhesive agents or coverings; anaesthetic or analgesic agents/s. SAP or some other applicable swelling materials known in the art may be contained within the absorbent patch to swell and increase the bulkiness thereof upon absorbing of fluids, such as blood, thereby facilitating an enhanced counterpressure against the area of biological tissue surrounding the perforation. Coagulating and/or vasoconstrictive agents, the latter include the examples of angiotensin and vasopressin, or any other of numerous suitable substances known in the art, may be contained within the absorbent patch to biochemically modify the blood or the surrounding biological tissue, thus intrinsically reducing the haemorrhage. Antiseptic, antibacterial or antifungal agents include numerous examples of suitable substances known in the art that are capable to prevent or reduce a bacterial or fungal infection. Anaesthetic or analgesic agents can be any of the numerous suitable substances known in the art that provide a relief from the pain and discomfort that may be experienced by the injured. Anti-adhesive agents or coverings preventing patch's adhering to the biological tissue surrounding the perforation include the example of TELFA™ Island Dressings, item code 7665, available from Tyco Healthcare/Kendall, 15 Hampshire Street, Mansfield, Mass. US.

According to the best mode of carrying out the invention, in some embodiments, the expandable flange and/or sealing element and/or at least the distal portion of the catheter member are made of biodegradable materials, thereby providing for suturing these components into the living tissues of the patient and remaining to degrade therein.

The expandable flange, as well as the catheter member, can assume a variety of forms, shapes and sizes and be made of various materials, all in order to suit the specific implementation of the DSPSFL and comply with the requirements of the medical condition to be treated. Thus, for instance, the size can vary in order to cover a range of perforations of different sizes and/or to comply with the anatomical constraints associated with different body parts to be treated or in order to accommodate different types of injuries, perforations, raptures or incisions.

In view of the foregoing, it is stressed that all the variations of the expandable flange and/or the catheter member, regardless of their particular form, shape, size, materials they are made of are considered to be within the scope of the present invention, provided that the expandable flange is located at the distal end of the catheter member and inserted through a perforation rapture or incision into a organ to be treated/sealed, wherein the expandable flange assumes its expanded sprawled or unfolded conformation.

The sealing element can take a variety of forms, shapes and sizes and can be made of various materials, all in order to suit the specific implementation of the device and comply with the requirements of the medical condition to be treated. Thus, for instance the size of the sealing element can vary in order to cover a range of perforations of different sizes and or to suit the anatomic sites to be treated with the device and the shape can vary in order to suit different kind of injuries.

In view of the foregoing, it should be stressed that all the variations of the sealing element, regardless of their particular form, shape, size or materials they are made of, are considered to be within the scope of the present invention, provided that the condition that the sealing element is pressed against the expandable flange, onto the biological tissue surrounding the perforation, from the outside of the organ treated/sealed.

### OPERATION OF THE DEVICE OF THE INVENTION

It is noted that the method of operating the embodiment of DSPSFL adapted for sealing perforations in an essentially tubular organs, shown in FIGS. 1 to 4 and FIGS. 5 to 6, is explained below by the way of example of operating the DSPSFL dedicated for globular or apical organs, shown in FIGS. 7 to 8. Accordingly the references and numerals of particular parts or portions of the DSPSFL and/or the subject organ/s it is implemented on should be construed mutatis mutandis and interchangeably with their respective equivalents and counterparts of DSPSFL shown in FIGS. 1 to 6.

DSPSFL 300A is typically packed in a sterile packaging, while the cannula member (not shown) is anteriorly disposed on catheter member 310; thereby confining expandable flange 314A and preventing the sprawling/unfolding thereof, i.e. retaining it in the contracted or folded conformation. In the contracted or folded conformation the effective exterior diameter of DSPSFL 300A is the outer diameter of the cannula member (not shown) and the anterior end is essentially the tip of the confining sleeve (not shown), occluded by mandrel 312 inserted thereto and expandable flange 314A folded therein. The sealing element is disposed on the confining sleeve (not shown) towards the posterior portion of DSPSFL 300A.

In the instance of DSPSFL adapted for sealing perforations in essentially tubular organs, shown in FIGS. 1 to 4 and FIGS. 5 to 6, the concave interior surface of expandable flange 16 is preferably disposed inside the cannula vis-à-vis the pointed end of tip 32. The aforementioned orientation of the concave interior surface of expandable flange 16 is disposed inside the cannula vis-à-vis the pointed end of tip 32, provides for inserting the pointed end of tip 32 into the perforation first and then deploying expandable flange 16 so that the convex exterior face thereof facing the perforation.

The operator appends DSPSFL 300A to the perforation, rapture or incision to be sealed. If the perforation is formed within a deep wound, the operator may introduce the anterior portion of DSPSFL 300A, namely anterior portion of confining sleeve (not shown) thereto, until the tip of the cannula member (not shown) is put adjacently to the perforation, rapture or incision. The operator then, by manipulating posterior end of DSPSFL 300A and particularly the proximal cap of mandrel 312, inserts the tip of the cannula member (not shown) throughout the perforation or incision into the organ to be sealed. Provided that the organ to be sealed is a part of the cardiovascular system, the operator may refer to the blood indicator, to verify the presence of the tip of mandrel 312 in a positive blood pressure environment.

Following the insertion of the tip of the cannula member (not shown) operator deploys expandable flange 314A inside the organ to be sealed, by purposefully pulling the tab of the cannula member over catheter member 310 while stabilizing the posterior cap of mandrel 312. Upon pulling the tab of the cannula member (not shown), the confining sleeve (not shown) is posteriorly retracted and the tip of the cannula member is slid away over expandable flange 314A; thereby flange 314A and the anterior portion of the catheter member 310 are exposed. Consequently expandable flange 314A spontaneously unfolds/sprawls, thus assuming an expanded sprawled or unfolded conformation, such as flange 3148, inside the organ. To completed the deployment of expandable flange 3148 within the organ, the operator may slightly pull DSPSFL 300A in a posterior direction, so that the proximal face of the flange 314B, e.g. the convex face of expandable flange 16, adjoins an area of biological tissue surrounding the perforation from inside the organ, e.g. the concave face of interior of a blood vessel.

After expandable flange 3148 was deployed in the organ to be sealed by assuming an expanded sprawled or unfolded conformation therein, mandrel 312 can be removed by being posteriorly retracted and eventually pulled out of the interior lumen of catheter member 310. Subsequently clamp 330 can be compressed to prevent an outflow from the outlet of DSPSFL 300B. By applying a manual force to clamp 330, slidably driving it along catheter member 310, the sealing element is urged towards expandable flange 314B, so that an area of biological tissue surrounding the perforation is captured within gap 340, namely in-between flange 314B and the sealing element, as schematically shown in FIG. 7C. Inflatable toroidal cushion 318 is thence can be inflated facilitating an enhanced sealing of the perforation; simultaneously the inflation of toroidal member 318 affixes it to the exterior surface of catheter member 310.

Upon exposure to a positive pressure environment inside the organ, the thin-walled neck portion of the catheter member, such as neck portion 14, expands, thus adjoining the edges of the perforation or incision, thereby facilitating an enhanced sealing thereof; whereas the infinitesimal intrinsic bias of the thin-walled neck portion of the catheter member does not forcefully spreads the tissue of the perforation, thus providing for the closure thereof.

The inlet of DSPSFL 300C at proximal end of catheter member 310 can then be connected to a source of infusion medium, which can be perfused via the interior lumen of catheter member 310 into the organ through outlet 345 at the distal end thereof. The lumen of catheter member 310 allows sufficient amounts of liquids to pass through in a relatively short period of time and characterised by flow rates of at least 0.25-0.5 L/min.

### SCOPE OF USE OF THE OF THE INVENTION

The device of the present invention is inter alia intended for an initial emergency field-care, typically performed within the vicinity of the site and with proximity to the time of the injury incident occurrence in order to substantially reduce a haemorrhage, avoid exsanguination, and stabilize injurer's condition for further transportation to a medical facility. Nevertheless, the device's usage is not limited to emergency field-care of injury incidents, thus the device of the present invention is equally applicable for all perforations and raptures of the blood vessels and/or heart walls regardless the nature of the phenomena that caused such perforation or rupture, which is of a noticeable clinical benefit to the patient. Instances of such other perforations and ruptures include: a spontaneous rupture that may occur in the walls of a heart or abdominal aorta or iatrogenic trauma caused in a surgery room.

Additional application of the DSPSFL of the present invention is the performing of a quick aortic cannulation procedure, by obviating the purse-string sutures for affixing the cannula. Such rapid cannulation can be beneficially employed for achieving a cardiopulmonary bypass to heart-lung life support systems, as promptly as possible. If the DSPSFL is employed for aortic cannulation, the DSPSFL is typically deployed through an incision. The embodiments of the device to be used as aortic cannulae shall have accordingly designed expandable flange and sealing element. Shape, size and other physical properties of the expandable flange and sealing element may also vary to meet the requirements of implementing the device in a particular cannulation procedure or particular site of cannulation, such as ascending or descending aorta.

It should be acknowledged, however, that the implementation of DSPSFL is not limited to the cardiovascular system, e.g. blood vessels or the heart. The DSPSFL can be beneficially implemented of various organs and/or tracts and employed in various medical applications. Thus the DSPSFL can be beneficially implemented on any anatomic passage, cavity or tubuloalveolar structure formed within a body or an organ, such as the gastrointestinal tract, respiratory tract, urinary tract and male or female reproductive tract, including the esophagus, stomach, small intestine and duodenum colon, trachea, pleura/pleural cavity, lungs, urethra, ureters and bladder. The DSPSFL can also be employed as a trocar for draining liquids or gases from the sealed organ. An application of the DSPSFL as a trocar can be exemplified by intercostal tube thoracostomy treatment of pneumothorax.

The necessity in the device of the present invention can be emphasized by the long lasting unsatisfied need therefor; since hitherto physicians and paramedics occasionally used to improvise with Foley catheters in lieu of a dedicated device, in order to seal perforations occurred in the ventricles or atriums of a heart. However, Foley catheters are less then suboptimal for the task achieved by method and device of the present invention, since the size and the spherical shape of the inflatable balloon of the Foley catheter would completely block any blood vessel and fail to achieve an efficient anastomosis.

It will be appreciated that the present invention is not limited by what has been particularly described and shown hereinabove and that numerous modifications, all of which fall within the scope of the present invention, exist. Rather the scope of the invention is defined by the claims which follow:

1-24. (canceled)

25. A medical device for at least sealing perforations comprising:
(a) a catheter member comprising at least one expandable flange at the distal end thereof, said flange capable of assuming at least one conformation selected from the group consisting of: a contracted conformation, folded conformation, expanded conformation, sprawled conformation and unfolded conformation;
(b) a mandrel member, wherein said mandrel member is insertable into said catheter member;
(c) a cannula member, wherein said cannula member is disposable over said catheter member;
(d) a sealing element;
wherein said sealing element is threadable onto at least one aforesaid member of said medical device selected from the group consisting of: said catheter member and said cannula member.

26. A medical device as in claim 25, wherein said expandable flange retained in a conformation selected from the group consisting of: said contracted conformation and said folded conformation by said cannula member.

27. A medical device as in claim 25, wherein said catheter member further comprises a relatively thin-walled neck portion adjacently to said flange.

28. (canceled)

29. A medical device as in claim 25, wherein said sealing element comprises at least one element selected from the group consisting of: a bushing, toroidal cushion, inflatable lumen and a conduit.

30. A medical device as in claim 25, wherein said expandable flange is characterized by a trough-shaped configuration, wherein the convex surface thereof is adapted to adjoin the interior concave surface of an essentially tubular organ.

31. A medical device as in claim 25, wherein said expandable flange comprises an outlet aperture forming a continuum with an interior lumen of said catheter member.

32. A medical device as in claim 25, wherein said expandable flange comprises at least one structural element adapted for engagement with said mandrel member.

33. A medical device as in claim 25, wherein said mandrel member comprises at least one structural element adapted for engagement with said expandable flange.

34. A medical device as in claim 25, wherein said mandrel comprises a blood indicator.

35. A medical device as in claim 25, wherein said tip of said cannula member is characterized by at least one property selected from the group consisting of: a shape of a tip of an aortic cannula and size, of a tip of an aortic cannula.

36-51. (canceled)
